(19) United States
(12) Patent Application Publication
(10) Pub. No.: US 2010/0120063 A1
(43) Pub. Date: May 13, 2010

(54) GPCR Arrestin Assays

(75) Inventors: Daniel Bassoni, Campbell, CA (US); Keith R. Olson, Pleasanton, CA (US); Thomas S. Wehrman, Mountain View, CA (US)

Correspondence Address: PETERS VERNY , L.L.P., 425 SHERMAN AVENUE, SUITE 230, PALO ALTO, CA, 94306, US

(73) Assignee: DISCOVERX CORPORATION, Fremont, CA (US)

(21) Appl. No.: 12/577,175

(22) Filed: Oct. 10, 2009

Related U.S. Application Data
(60) Provisional application No. 61/104,374, filed on Oct. 10, 2008.

(51) Int. Cl. G01N 33/53

(52) U.S. Cl. 435/7.2

(57) ABSTRACT

Sensitive assays for candidate compounds affecting GPCR activity are provided using a cell containing fusion proteins comprising a first fusion protein comprising (a) a target GPCR fused to a small fragment of β-galactosidase through a linker comprising a phosphorylation site or (b) a GPCR or a protein of interest, where the GPCR and protein of interest form a complex and one of them is fused to the small fragment of β-galactosidase; and a second fusion protein comprising arrestin fused to a large fragment of β-galactosidase. In (a), the affinity of the small and large fragments is optimized based on the background to signal ratio and the absolute signal observed. The assay is performed using a β-galactosidase substrate that provides a detectable optical signal.

### CROSS-REFERENCE TO RELATED APPLICATIONS

This application claims priority from U.S. Provisional Application No. 61/104,374, filed on Oct. 10, 2008, which is hereby incorporated by reference in its entirety.

### STATEMENT OF GOVERNMENTAL SUPPORT

None.

### REFERENCE TO SEQUENCE LISTING

Applicants assert that the paper copy of the Sequence Listing is identical to the Sequence Listing in computer readable form found on the accompanying computer file. Applicants incorporate the contents of the sequence listing by reference in its entirety.

### BACKGROUND OF THE INVENTION

1. Field of the Invention

The present invention relates to the field of the evaluation of agonists or antagonists of G protein-coupled receptors.

2. Background

G protein coupled receptors (“GPCRs”) are a large class of seven transmembrane domain receptors that transduce signals from outside the cells when bound to an appropriate ligand. The GPCRs have a myriad of functions, being involved in sensory perceptions, such as odor and vision, responding to pheromones, hormones and neurotransmitters, where the ligands greatly vary in nature and size. The GPCRs can affect behavior and mood, the immune system, the sympathetic and parasympathetic nervous systems, cell density sensing and there may be additional physiological activities that involve GPCRs in their pathway. The GPCRs are associated with a number of diseases and have been an active target of pharmaceutical companies.

GPCRs are activated by an external signal resulting in a conformational change. It appears that once the receptor becomes bound it activates the G protein, which G protein is bound to ATP. The G protein is a trimer, which upon activation converts GTP to GDP. Active GPCRs are phosphorylated by protein-coupled receptor kinases. In many cases upon phosphorylation, the phosphorylated receptor becomes linked to arrestin. The binding to arrestin may result in translocation of the GPCR or other outcome.

GPCRs can exist as monomers, dimers, or heterodimers, when expressed in mammalian cells. The ability of GPCRs to form heterodimers provides a novel mechanism of cellular signaling. Two GPCRs that heterodimerize or one GPCR and a receptor that binds to the GPCR can attain signaling functions and ligand binding functions that are distinct from when only one of the receptors is present in a cell. As indicated above, the GPCRs are important to the functioning of a cell. Where the GPCR activation results in the regulation of another GPCR expressed on the same cell, there is interest in being able to detect and modulate the dimer- or oligomerization. By inhibiting the complexing of the GPCR with another membrane protein necessary for signal transduction, one can affect the pathway(s) regulated by the GPCR and the pathway(s) affected by the second membrane protein. There is substantial interest in determining the effect of ligand binding to a GPCR, as well as heterodimeric GPCR complex on cell pathways.

In view of the importance of the GPCRs on the physiological status of mammals, there has been substantial interest in developing compounds that can modulate the activity of specific GPCRs and the interaction of GPCRs with other proteins in the cellular membrane and in the cytosol. As part of this process, compounds are screened as to their ability to induce the binding of arrestin to the GPCRs. One technique that has been employed to assay the effect of a candidate ligand is enzyme fragment complementation (“EFC”), where the two enzyme fragments may be fused to two different proteins. When the two proteins complex, the two enzyme fragments are brought together to form an active enzyme. This technique has been exploited in U.S. patent application nos. 2007/0275397; 2005/0287522; and 2003/0175836. However, when this methodology was applied to the complexing of GPCRs and arrestin to determine the effect of a candidate ligand, in many cases a weak or no signal was observed. Because of the versatility and sensitivity of the system one obtains amplification from the formed β-galactosidase (there, there is substantial interest in adapting EFC to evaluating candidate ligands.

### RELEVANT LITERATURE

See, the U.S. patent applications indicated above. U.S. Pat. No. 7,235,374 describes mutant GPCRs incorporating serines and/or threonines in the C-terminal region of the GPCR and using β-galactosidase fragments for detection. See also as illustrative of activity in the field, Hammer, et al. 2007, FASEB J. 21, 3827-34; Molinari, et al. 2008, Biochem. J., 409, 251-61; Hamadan, et al. 2007, J. Biol. Chem., 282, 29089-100; Garippa, et al. 2006, Methods Enzymol., 414, 99-120; and Yan, et al. 2002, J. Biomol. Screen., 7, 451-9.

### SUMMARY OF THE INVENTION

The following summary is not intended to include all features and aspects of the present invention, nor does it imply that the invention must include all features and aspects discussed in this summary.

Systems are provided for detecting the binding of a ligand to a GPCR or GPCR heterodimer resulting in transduction of a signal. Depending on whether one is measuring binding of a ligand to a GPCR or to a member of a GPCR heterodimer with other than a G protein or arrestin, different approaches are employed. One may engineer cells to express one type of GPCR, in which case no heterodimer is formed (i.e. no “protein binding partner” is present), or one may engineer cells to express two complexing types of GPCR.

A cell based tunable system for measuring the effect of a candidate compound on signal transduction is employed. Genetic constructs are prepared where the binding affinities of the GPCR and arrestin and the members of β-galactosidase enzyme fragment complementation (“EFC”) pair are selected to provide a robust assay. To enhance binding of arrestin to the GPCR, a member of the EFC pair may be fused to an intact GPCR at its C-terminus through a phosphorylation linker having at least one phosphorylation site to enhance arrestin binding and the binding affinities of the EFC pair varied in accordance with the binding affinities of the GPCR and arrestin to provide for a robust signal and a large signal to background ratio. The gene encoding arrestin is fused to the gene encoding the other fragment of the β-galactosidase enzyme fragment complementation pair. Genetic constructs are employed for introduction into the cells under conditions for expression and a substrate providing for a detectable product added after sufficient time for arrestin to bind to the GPCR.

In certain aspects, the present invention comprises the use of fusion proteins of an enzyme fragment to a GPCR or a GPCR binding protein. GPCR binding proteins are exemplified below as different CPCRs from the CPCR being activated by a ligand. The activated GPCR forms a heterodimer with the binding protein. As indicated previously, GPCRs can exist as heterodimers, where two different GPCRs or a GPCR and a different receptor form a heterodimer. To determine whether there is this type of transactivation, a member of the EFC pair may be fused to either protein, where the ligand is selected so as not to bind to a GPCR fused to a member of the EFC pair. Where ligand binding to one of the members of the heterodimer results in the other member, a GPCR, complexing with an arrestin fused to the gene encoding the other fragment of the β-galactosidase enzyme fragment complementation (“EFC”) pair. For more details, see Prinster et al., “Heterodimerization of G Protein-Coupled Receptors: Specificity and Functional Significance,” Pharmacol Rev 57:289-298, 2005. As reported there, there are at least three distinct ways that GPCR heterodimerization may be physiologically relevant. First, some GPCRs are completely nonfunctional when expressed alone and clearly require assembly with a specific partner to achieve surface expression and functional activity. Second, even GPCRs that do not absolutely require heterodimerization may still associate with other receptors, allowing for cross talk and mutual regulation between specific receptor subtypes. Third, GPCR heterodimerization can in some cases alter the pharmacological properties of the associated receptors, such that novel pharmacological entities are created. Heterodimerization between closely related members of the GPCR family has been observed for GABABR1-GABABR2; M2M3 muscarinic; {kappa}-{-delta} opioid; μ-{delta} opioid; 5HT1B-5HT1D serotonin; SSTR1-SSTR5 somatostatin; SSTR2-SSTR3 somatostatin; and CCR2-CCR5 chemokine receptors. Thus, with regard to one of these specific GPCRs, one of its possible different complexing partners may be regarded as a protein binding partner.

The use of arrestin affords the use of the present assay with a wide variety of GPCRs. In one embodiment, the larger portion of beta galactosidase, the EA is fused to the C-terminus of beta arrestin. The ED, about 4 kDA, is expressed as a fusion protein with the GPCR of interest, at the C terminus. Upon activation the arrestin binds to the GPCR and an active beta galactosidase is formed.

The β-galactosidase is measured in accordance with conventional procedures using fluorescence or chemiluminescence.

### BRIEF DESCRIPTION OF THE FIGURES

FIG. 1A is a graph of results, tabulated below, of the screening assay described in the Experimentals section below for the GPCR receptor CHRM2 when fused to the mutated ED (PK1) (SEQ ID NO:11). PK1 gave a 1.4 fold response. FIG. 1B is a graph of the results, also tabulated below, for the GPCR receptor CHRM2 when fused to the wild-type ED (PK2) (SEQ ID NO:12). PK2 enhances signal to background for receptors that interact weakly with arrestin. The ligand oxotremorine is used. Oxotremorine is a synthetic alkaloid and is a muscarinic agonist in that it will bind to muscarinic acetylcholine receptors.

| | S:B | S:B |
|---|---|---|
| Receptor | PK1 | PK2 |
| CHRM2 | 1.6 | 6.1 |

FIG. 2 is a graph of screening assay results, tabulated below, showing a comparison of results with the GPCR receptor SSTR1, where no response was obtained with PK1 and a robust response was obtained with PK2. SSTR1 refers to the somatostatin receptor 1. It is known that The use of Tyr1[d-Trp8]somatostatin as a labeled ligand permits accurate determinations of the binding affinity and concentration of receptors for somatostatin in the normal pituitary gland.

| | CHO Arrestin + SSTR1-PK2 |
|---|---|
| BOTTOM | 4795 |
| TOP | 10484 |
| LOGEC50 | −8.089 |
| HILLSLOPE | 0.7893 |
| EC50 | 8.1506e−009 |

FIG. 3 is a graph showing a comparison of screening assay results, tabulated below, obtained with the GPCR receptor CRTH2 and PK1 vs those obtained with CRTH2 and PK2 where no response was obtained with PK1 and a robust response was obtained with PK2. CRTH2, a prostaglandin receptor, was exposed to prostaglandin.

| | CHO Arrestin2 + CRTH2-PK2 |
|---|---|
| BOTTOM | 29223 |
| TOP | 75978 |
| LOGEC50 | −6.760 |
| HILLSLOPE | 1.018 |
| EC50 | 1.7376e−007 |

FIG. 4 is a graph showing comparison of screening assay results, tabulated below, obtained with the GPCR receptor CHRM2, where no response was obtained with PK1 and a robust response was obtained with PK2. Oxotremorine is used as an agonist as in FIG. 1.

| | CHO Arrestin + CHRM2-PK2 |
|---|---|
| BOTTOM | 335.9 |
| TOP | 2037 |
| LOGEC50 | −5.201 |
| HILLSLOPE | 1.183 |
| EC50 | 6.2881e−005 |

FIGS. 5A-D show a series of four graphs of assay performance with the GPCR receptors MC1R (A-B) and HRH1 (C-D) in the presence and absence of the linker. FIG. 5A shows a graph of screening assay performance with the GPCR receptor MC1R in the absence of the EGS inker. FIG. 5B shows a graph of screening assay performance with the GPCR receptor MC1R in the presence of the EGS linker. FIG. 5C shows a graph of screening assay performance with the GPCR receptor HRH1 in the absence of EGS the linker. FIG. 5D shows a graph of screening assay performance with the GPCR receptors HRH1 in the presence of the lEGS inker

FIG. 6 is a graph of screening assay results, tabulated below, obtained with the cell line U2OS, the GPCR receptor SSTR4 and the EGS linker having multiple serine phosphorylation sites, and PK where there was no response in HEK cells without the linker and 1.5× response in CHO cells.

| | U208 A2 S ST R4 |
|---|---|
| BOTTOM | 1380 |
| TOP | 3418 |
| LOGEC50 | −9.136 |
| HILLSLOPE | 2.138 |
| EC50 | 7.3187e−010 |

FIG. 7 is a graph showing comparison of results (tabulated below) obtained with the receptor HRH3 in the presence and absence of the EGS linker, where there was no response in the absence of the linker.

| | CHO Arrestin2 + HRH3 |
|---|---|
| BOTTOM | 273.1 |
| TOP | 934.9 |
| LOGEC50 | −7.258 |
| HILLSLOPE | 0.5419 |
| EC50 | 5.5194e−008 |

FIG. 8 is a graph showing a comparison of screening assay results, tabulated below, obtained with the receptor CHRM3 in the presence and absence of the EGS linker, where there was no response in the absence of the linker.

| Best-fit values | CHO A2 CHRM3-egs |
|---|---|
| BOTTOM | 545.9 |
| TOP | 1546 |
| LOGEC50 | −7.085 |
| HILLSLOPE | 1.056 |
| EC50 | 8.2228e008 |
| | T/B = 3.0 |

FIGS. 9A-B together provide diagrams of the construction of the PK fusion protein plasmid vector. FIG. 9A shows the parental vector showing restriction sites AvrII-1462-C′CTAG_; HindIII-1492-A′AGCT_T and NOT I-1671-GC′GGCC_GC. The ONeo, GPCR and Amp genes are indicated by the arrows. FIG. 9B shows the finished GPCR-PK fusion plasmid showing restriction sites HindIII 2821_A′AGCT_T and NOT-3000 GC′GGHCC_GC. The ONeo, GPCR and Amp genes are indicated by the arrows.

FIGS. 10A-D provides a series of four graphs showing the specificity of the subject assay for the CCK8 ligand binding to the fusion protein CCKAR-PK. In FIG. 10A, CCK8 is added to a cell line (HEK) expressing CCKAR-PK, and arrestin-EA, and the effects on calcium concentration are measured. In FIG. 10B, CCK8 is added to a cell line (HEK) expressing CCKAR-PK, and arrestin-EA, and the effect on arrestin-EA binding is measured. In FIG. 10C, carbachol is added to an HEK cell line expressing CCKAR-PK and arrestin EA, and the effect on calcium concentration is measured. In FIG. 10D, carbachol is added to an HEK CCKAR-PK arrestin cell line and the effect on arrestin binding is measured. It is noted that the calcium increase resulting from the binding of CCK8 to CCKAR parallels the result observed for the arrestin-EA binding to the CCKAR-PK. However, for the non-ligand carbachol, while there is some effect on calcium increase, there is substantially no effect on the binding of arrestin-EA to CCKAR-PK.

FIG. 11A shows the results, tabulated below, of treating U2OS cells expressing OPRM1-PK with a negative ligand, deltorphin;

| | U2OS A2 OPRM1 + D1 |
|---|---|
| BOTTOM | 722.5 |
| TOP | 14441 |
| LOGEC50 | −3.123 |
| HILLSLOPE | 0.8348 |
| EC50 | 0.0007 |
| | [S:B = 2.5] |

FIG. 11B shows the results of treating U2OS cells expressing OPRM1-PK and OPRD1 with an agonist, deltorphin, for OPRD1.

| | U2OS A2 OPRM1 + D1 |
|---|---|
| BOTTOM | 613.9 |
| TOP | 5380 |
| LOGEC50 | −8.077 |
| HILLSLOPE | 1.224 |
| EC50 | 8.3757e009 |
| | [S:B = 7.4 |

FIG. 12A shows the results, tabulated below, of treating CHO cells expressing OPRD1-PK with an OPRM1 agonist

| | CHO A2 OPRD1 + M1 |
|---|---|
| BOTTOM | 519.8 |
| TOP | 2809 |
| LOGEC50 | −4.439 |
| HILLSLOPE | 1.293 |
| EC50 | 3.6394e |

FIG. 12B shows the results, tabulated below, of treating U2OS cells expressing OPRM1-PK with an OPRM1 agonist;

| | CHO A2 OPRD1 + M1 |
|---|---|
| BOTTOM | 1008 |
| TOP | 10875 |
| LOGEC50 | −3.872 |
| HILLSLOPE | 0.7069 |
| EC50 | 0.000134 |

FIG. 12C shows the results, tabulated below, of treating CHO cells co-expressing OPRD1-PK and OPRM1, where there is no observable transactivation;

| | CHO A2 OPRD1 + M1 |
|---|---|
| BOTTOM | 20.31 |
| TOP | 201.4 |
| LOGEC50 | −6.949 |
| HILLSLOPE | 1.571 |
| EC50 | 1.1235 |
| | [S:B = 31.9] |

FIGS. 13A-D is a series of four graphs where graphs A and B show the results of a screening assay obtained by treating of U2OS cells co-expression of OPRM1-PK and CCR2 (top) and OPRM1-PK and CCR5 with CCL2 or CCL3, respectively. The results are compared to cells lacking the CCR2 and CCR5 receptors, respectively. The graphs C and D show the response of HEK cells expressing CCR2-PK and CHO cells expressing CCR5-PK when treated with CCL2 and CCL3, respectively; and

FIG. 14A shows the results, tabulated below, of treating of U2OS cells expressing OPRM1-PK with the agonist DADLE.

| | OPRM1 + PRC |
|---|---|
| BOTTOM | 625.6 |
| TOP | 17070 |
| LOGEC50 | −6.954 |
| HILLSLOPE | 1.483 |
| EC50 | 1.1124 |
| | [S:B = 32.7] |

FIG. 14B shows the results, tabulated below, of treating U2OS cells expressing OPRM1-PK with the agonist EG-VEGF.

| | OPRM1 + PRC |
|---|---|
| BOTTOM | 25.82 |
| TOP | 57.34 |
| LOGEC50 | −7.493 |
| HILLSLOPE | 1.011 |
| EC50 | 3.2105e−008 |

FIG. 14C shows the results of treating U2OS cells co-expressing OPRM1-PK and PROKR2, with EG-VEGF, the agonist for PROKR2. (Note A2 and arrestin refer to the presence of arrestin-EA fusion protein.)

| | CHO Arrestin2 + PROKR2 |
|---|---|
| BOTTOM | 294.3 |
| TOP | 9875 |
| LOGEC50 | −7.282 |
| HILLSLOPE | 1.328 |
| EC50 | 5.2250e−008 |

### DESCRIPTION OF THE SPECIFIC EMBODIMENTS

Methods and compositions are provided for determining the effect of a candidate compound on the transduction of a signal as a result of binding of arrestin to G-protein coupled receptors (“GPCRs”). The subject methods allow for measurement of ligand to a GPCR, complex formation of a GPCR with a protein of interest, and the effect of a candidate compound on these events. Different genetic constructs are provided for the different measurements, where either the GPCR or the protein of interest is fused to a member of the β-galactosidase enzyme fragment complementation (“EFC”) pair.

Generally speaking, the subject invention provides a method for screening binding of a GPCR to at least one of a GPCR ligand or a protein of interest by employing a β-galactosidase enzyme fragment complementation assay, using an enzyme donor fragment (“ED”) and an enzyme acceptor fragment (“EA”). Exemplary E. coli β-galactosidase sequences include those described at GenBank accession numbers AAN78938, ABI99820 and A7Z191.

Employed in the method are a first fusion protein comprising (a) a GPCR linked to a fragment of β-galactosidase (“ED”) optionally joined to a sequence comprising a naturally occurring GPCR phosphorylation site or a consensus sequence of naturally occurring GPCR phosphorylation sites, exemplifed below as “EGS”, which links to an enzyme donor fragment (“ED”) or (b) a protein of interest or a GPCR joined to an ED. (Usually, ED is the small fragment.) In the case of (a) ligand binding to a GPCR is measured. In the case of (b) a protein of interest binding to a GPCR in the presence of a ligand that binds the protein of interest is measured, with the caveat that the ligand does not bind to a GPCR fused to ED. Also employed is a second fusion protein comprising arrestin linked to the complementary fragment of β-galactosidase (“EA”), where when said arrestin is bound to said GPCR a functional β-galactosidase is formed. For (a) the ED, EA and presence of said linker are selected to provide binding of said GPCR to arrestin to provide a substantially optimized signal. For the most part, mammalian cells are transformed with the genetic constructs expressing the first and second fusion proteins. In performing the method, the cells are incubated in an assay medium in a selected environment, normally including an agonist or agonist candidate, for sufficient time for any binding to occur, followed by the addition of a β-galactosidase substrate, which substrate results in a detectable signal, and then determining the signal as a measure of the binding. In some instances, one may study an antagonist for displacing the agonist or a compound modulating the binding of the GPCR to the protein of interest.

The methods employ genetically modified cells, where the cells are modified with first and second genetic expression constructs. The method employs enzyme fragment complementation (“EFC”) with β-galactosidase to detect the binding of arrestin to a GPCR. The method relies on a compound binding to a GPCR where arrestin fused to a member of the β-galactosidase enzyme fragment complementation (“EFC”) pair binds to the GPCR.

In a first embodiment, the method relies on tuning the factors that affect the binding affinity of the expression products, by properly selecting the EFC members as to their affinity for complexing to form an active enzyme and optionally modifying the intact GPCR to enhance its affinity for arrestin. An absolute signal and a signal to background ratio can be obtained that provides for sensitive detection of the arrestin binding to the GPCR. The binding of arrestin to the GPCR is detected using a β-galactosidase substrate that produces a detectable signal.

In a second embodiment, the method relies on the existence of a protein of interest and a GPCR being bound together for activation and signal transduction, where an ED is fused to one of the proteins and a ligand is employed to bind to one of the proteins, with the restriction that the ligand will be selected so as to not bind to a GPCR when fused to the ED. Transactivation results in the activation of the GPCR, where the arrestin-EA fusion binds to the transactivated GPCR.

In the first embodiment, the first construct comprises a gene encoding for a GPCR linked to a β-galactosidase fragment, conveniently the small fragment of β-galactosidase (“ED” or “PK”), optionally through a linker comprising at least one phosphorylation site. The genetic construct is under the control of a transcriptional and translational regulatory region functional in the cellular host. The second construct comprises a gene encoding for arrestin fused to the other fragment of β-galactosidase under the control of a transcriptional and translational regulatory region functional in the cellular host. The genetic constructs are introduced into an appropriate mammalian cell host under transient or permanent conditions for expression of the constructs. The host is able to respond to a candidate ligand binding to the GPCR, where the host provides the ancillary components for arrestin to bind to the GPCR, e.g., the proper G-protein.

In the second embodiment a first construct comprises a gene encoding for a protein of interest or a GPCR fused to a β-galactosidase fragment, conveniently the small fragment of β-galactosidase (“ED” or “PK”). The genetic construct is under the control of a transcriptional and translational regulatory region functional in the cellular host. The second construct comprises a gene encoding for arrestin fused to the other fragment of β-galactosidase under the control of a transcriptional and translational regulatory region functional in the cellular host. If desired, the GPCR may be modified with the linker described below to enhance the affinity with arrestin. The genetic constructs are introduced into an appropriate mammalian cell host under transient or permanent conditions for expression of the constructs. The host is able to respond to a candidate compound that affects complex formation between the protein of interest and a GPCR.

The system is very versatile in the many different modes in which it can be performed, where a GPCR complexes with another protein other than arrestin and the G proteins. The other protein can be a protein of interest located in the cell membrane or the cytosol, so long as the other protein has access to complexing or interacting with the GPCR. This embodiment can be used to study ligands for the GPCR, ligands as agonists or antagonists, where the GPCR fusion with a β-galactosidase fragment may be undesirable. The embodiment can also be used to screen proteins to determine whether they bind to a GPCR and the extent to which they bind. Alternatively, one can measure candidate compounds for their effect on the interaction between the protein of interest and the GPCR, other than as a ligand for the GPCR. By using different GPCR ligands that activate different GPCRs and a target GPCR fused to ED, one can screen for which GPCRs interact with the target GPCR. Also, with a receptor protein of interest that recruits and activates a GPCR upon agonist binding, one can screen candidate compounds that act as agonists or antagonists. In addition, to the extent that a protein of interest forms a heterodimer with a GPCR, one can also study candidate compounds that modulate such heterodimerization.

As is known, arrestin exists in different species, and, in humans, in different homologs, such as ARRB2 and ARRB1. Using a low stringency hybridization technique to screen a rat brain cDNA library, Attramadal et al. isolated cDNA clones representing 2 distinct beta-arrestin-like genes. One of the cDNAs is the rat homolog of bovine beta-arrestin (beta-arrestin-1; ARB1; 107940). In addition, Attramadal et al. isolated a cDNA clone encoding a novel beta-arrestin-related protein, which they termed beta-arrestin-2. ARB2 exhibited 78% amino acid identity with ARB1. The primary structure of these proteins delineated a family of proteins that regulate receptor coupling to G proteins. ARB1 and ARB2 are predominantly localized in neuronal tissues and in the spleen. See Attramadal, H.; Arriza, J. L.; Aoki, C.; Dawson, T. M.; Codina, J.; Kwatra, M. M.; Snyder, S. H.; Caron, M. G.; Lefkowitz, R. J., Beta-arrestin-2, a novel member of the arrestin/beta-arrestin gene family. See J. Biol. Chem. 267: 17882-17890, 1992 for further information on this gene and protein. The beta arrestin amino acid sequence is given at world wide web address uniprot.org/uniprot/P49407#P49407-1.

When the protein of interest is in the cellular membrane, one can decide as to which of the proteins in the complex should be fused to the ED. When the protein of interest is in the cytosol, then the protein of interest will be fused to the ED. In the complex, when the ED is fused to the GPCR, then the arrestin-EA and ED will be in close proximity to form the functional enzyme. When the ED is fused to the protein of interest, then the EA will be brought by the arrestin binding to the GPCR in close proximity to the ED to form the functional enzyme. One chooses the preferred configuration depending upon the proteins involved in the assay, the observed signal, ease of construction, the purpose of the assay, and the like.

One can transform cells to be used in an assay with the genetic construct expressing the fusion protein of an arrestin-β-galactosidase fragment. Also, if the GPCR to be studied is not present in the cell, a construct expressing the GPCR may also be introduced in the cell. These cells could then be used with any protein of interest-β-galactosidase fragment to screen for complex formation and/or the effect of candidate compounds on complex formation between the protein of interest and the GPCR. Alternatively, one can prepare cells having a protein of interest-β-galactosidase fragment fusion construct and the arrestin-β-galactosidase fragment fusion construct for screening GPCRs that complex with the protein of interest.

The assay is performed under conventional conditions. Depending upon the mode of the assay different selected environmental conditions will be employed. For studying ligands for a GPCR, the selected environment will include a candidate ligand for the GPCR to detect any resulting activity, which results in the complex of the GPCR and arrestin. For determining complex formation, a GPCR or protein of interest agonist will be employed and for candidate compounds that modulate complex formation between a protein of interest and a GPCR, the candidate compound will be included. After sufficient incubation time for the arrestin to be transported and bind to the GPCR, β-galactosidase substrate is added and the turnover of the substrate determined, where the substrate provides for a detectable product. If desired, the cells are lysed and the substrate added with or after the addition of the lysing reagent. The complex of arrestin and the GPCR is sufficiently stable as to be retained after lysis, while the free arrestin in the lysate does not bind to the GPCR to any substantial degree. The resulting signal is a measure of the activity of the candidate ligand.

One component of the subject invention for many GPCRs is the linker between the GPCR and the small fragment of β-galactosidase (“ED”). The linker component will have at least one phosphorylation site (“phosphorylation linker”), desirably recognized by the same enzyme that phosphorylates the GPCR, namely G-receptor kinase (“GRK”). For the most part, the linker will have at least one S or T, usually between two and four and be of the general structure XZXₙZX, where n is from 1 to 3, usually 1, Z is S or T and X may be any amino acid other than S or T, usually an aliphatic amino acid, such as G, A, V, L, and I, the hydrocarbon side chain amino acids, aromatic amino acids, e.g., F, Y, and W, and basic amino acids, K and R. Desirably, the phosphorylation linker may have more than one phosphorylation site. The linker will either be a consensus sequence based on known GPCR phosphorylation sites or a naturally occurring GPCR phosphorylation site, which may have up to a total of about 3 modifications, including deletions, insertions and substitutions, the resulting modified sequence having ±3 nucleotides difference from the original sequence. The sequence may be synthesized based on a review of the consensus sequences of the GPCRs. Generally the phosphorylation linker will have at least about 5, more usually at least about 6, generally at least about 8, amino acids, and as a matter of convenience, not more than about 30, usually not more than about 25, generally not more than about 20, amino acids. There will generally be at least 2 aliphatic amino acids having hydroxyl groups, namely S and T, more usually at least 3, and there may be 10 or more, frequently the majority being S. The sequence may be the same as the phosphorylation site present in the GPCR to which the linker is attached or may be different.

Exemplary sequences in linkers that find use in the present invention include:

| | |
|---|---|
| GGGSGGGSLE; | (SEQ ID NO: 1) |
| SYNGSKXSPASLSRFS;; | (SEQ ID NO: 2) |
| SASYXSGHS; | (SEQ ID NO: 3) |
| CASLSRFSYSHYMS; | (SEQ ID NO: 4) |
| IASLSRLSYTTIS; | (SEQ ID NO: 5) |
| SQRSCSQPS | (SEQ ID NO: 6) |
| RSLXSCS; | (SEQ ID NO: 7) |
| DDSGSCLS; | (SEQ ID NO: 8) |
| SYSHMSAS; | (SEQ ID NO: 9) |
| SYTTISTL; | (SEQ ID NO: 10) |
| etc. | |

The exemplified EGS sequence is SEQ ID NO: 2, where X is serine. In addition to the phosphorylation linker, it is common to include a flexible linker to provide flexibility. Such linker may be a (polyG)S, where the number Gs will usually be in the range of about 3-6. Usually, the flexible linker will be proximal to the GPCR, although in some instances it may be proximal to the β-galactosidase fragment.

In preparing the construct of the GPCR, in the direction of translation (5′-3′), normally the intact gene will be fused to the flexible linker, if present, which in turn is fused to the phosphorylation linker, if present, which in turn is fused to the enzyme fragment.

The small fragment of β-galactosidase (“ED”) may have the naturally occurring sequence or a mutated sequence. Of particular interest are small fragments of from about 36 to 60, more usually not more than 50, amino acids. Desirably, the ED has a low affinity for the large fragment of β-galactosidase (“EA”), so that there is little complexation between the large and small fragments in the absence of binding of GPCR and arrestin, that is, the signal observed with the small fragment is at least about 50%, more usually at least about 70%, less than the signal observed with the commercially available fragment of 90 amino acids, when the two fragments are combined in the absence of fusion with other proteins. For further description of the small fragments, see U.S. Pat. No. 7,135,325. For further description of mutated EDs, see U.S. patent application publication no. 2007/0275397, both of which references are incorporated herein in their entirety as if set forth herein. The mutated ED will desirably have less than about 0.5, but at least about 0.1, of the activity of the wild-type sequence in the assay of interest or an analogous assay. For increasing affinity between the ED and EA, the longer EDs will be used and free of mutations from the wild-type sequence.

It is found that the combination of the linker and the ED are involved with the binding affinity of the modified arrestin to the modified GPCR. The system has a number of different affinities between components, some of which can be varied by the choice of groups. The natural affinity between the GPCR and arrestin is fixed as to a particular GPCR. The affinities between the different GPCRs and arrestin vary over a number of magnitudes. Providing the phosphorylation linker of the subject invention can increase the affinity between the GPCR and arrestin. However, the affinity may still be relatively low. The observed signal is dependent upon the level of binding of arrestin to GPCR upon stimulation of the GPCR. On the other hand, increased binding of arrestin to the GPCR in the absence of stimulation enhances the background signal, degrading the sensitivity of the assay. The affinity of ED for EA can be varied by appropriate choice of the ED. Longer EDs have greater affinity as compared to shorter EDs and mutated EDs, with the latter still providing a functional enzyme. By appropriate choice of ED, one can achieve an affinity that will optimize the assay sensitivity, providing lower background while still providing a robust signal. One can determine the choice of ED in relation to the affinity of the arrestin to the GPCR, using EDs of greater affinity for EA where the arrestin affinity for the GPCR is lower and EDs of lesser affinity, when the affinity for the arrestin is higher. To provide the optimal affinity one can select natural sequences or synthetic sequences having phosphorylation sites as described previously and then adapt the ED affinity for the EA to optimize the assay. This can be readily done empirically. It is found that in most cases, the signal to background ratio is more important than the absolute signal.

For the preparation of the fusion protein and its expression construct, conventional splicing and insertion techniques are employed. The ED will usually be linked to the C-terminus of the GPCR. The ED will come from the N-terminus proximal region of the β-galactosidase enzyme.

The fusion proteins provide a functional protein that is soluble, does not aggregate so as to be unavailable for complexing, has substantially the natural folding, so as to be susceptible to binding to endogenous proteins that normally complex to the polypeptide fused to the ED, and will usually be able to perform substantially the same functions that such polypeptide performs. Therefore, the polypeptide is capable of acting as a surrogate for the natural protein to allow for measurements that are predictive of the activity of the natural protein.

The ED may be joined to the coding region of the GPCR in a variety of ways. For a cDNA gene, one may select a suitable restriction site for insertion of the sequence, where by using overhangs at the restriction site, the orientation is provided in the correct direction. By using a plasmid in yeast having the cDNA gene, with or without an appropriate transcriptional and translational regulatory region, one may readily insert the ED construct so as to be fused to the linker(s) and the cDNA gene at an appropriate site.

Various conventional ways for inserting encoding sequences into a gene can be employed. For expression constructs and descriptions of other conventional manipulative processes, See, e.g., Sambrook, Fritsch & Maniatis, “Molecular Cloning: A Laboratory Manual,” Second Edition (1989) Cold Spring Harbor Laboratory Press, Cold Spring Harbor, N.Y. (herein “Sambrook et al., 1989”); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins EDs. (1985)]; “Transcription And Translation” [B. D. Hames & S. J. Higgins, EDs. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Any eukaryotic cell may be employed, for the most part cell lines being employed. The cell lines will usually be mammalian, but for some purposes unicellular organisms or cells from non-vertebrates can be used. Mammalian cell lines include CHO, HeLa, MMTV, HepG2, HEK, and the like. The cells are genetically modified transiently or permanently, usually permanently. Various vectors that are commercially available can be used successfully to introduce the two expression constructs into the eukaryotic cell. For an extensive description of cell lines, vectors, methods of genetic modification, and expression constructs, see published US application serial no. 2003/0092070, Zhao, et al., May 15, 2003, paragraphs 00046-00066, which are specifically incorporated herein by reference.

Transformed cells are cloned that have various expression levels of the fusion proteins. The best clone is then chosen by lowest EC50 and best signal to background ratio. The cells may be transiently or permanently transformed, in the case of the former using a conventional vector, normally a viral vector, e.g., adenovirus. Methods include transfection, electroporation, microinjection, transduction, cell fusion, DEAE dextran, calcium phosphate precipitation, lipofection (lysosome fusion), use of a gene gun, using a viral vector, with a DNA vector transporter, and the like. For permanent insertion into the genome, various techniques are available for the insertion of the sequence in a homologous or non-homologous fashion. These techniques are well known. For random insertion, the introduction of the nucleic acid by any of the above methods will usually be sufficient. For homologous recombination, see, for example, U.S. Pat. Nos. 7,361,641, 5,578,461, 5,272,071 and PCT/US92/09627, and references cited therein.

Regulatory regions that may be used will be functional in the cell and may be obtained from cellular or viral genes. Illustrative regulatory regions include many promoters that are commercially available today. Expression of the fusion protein may be controlled by any promoter/enhancer element known in the art, but these regulatory elements must be functional in the host or host cell selected for expression. Promoters which may be used to control fusion gene expression include, but are not limited to, the SV40 early promoter region (Benoist and Chambon, 1981, Nature, 290:304-310), the promoter contained in the 3′ long terminal repeat of Rous sarcoma virus (Yamamoto, et al., 1980, Cell, 22:787-797), the herpes thymidine kinase promoter (Wagner et al., 1981, Proc. Natl. Acad. Sci. U.S.A., 78:1441-1445), the regulatory sequences of the metallothionein gene (Brinster et al., 1982, Nature, 296:39-42), etc.

The screening method involves growing the cells in an appropriate medium and then washing the cells with an appropriate buffered aqueous solution, e.g., PBS. The cells are then incubated in serum-free medium, usually at least about 6 h, preferably at least about 12 h, where shorter times appear to degrade performance in some cases. Following the incubation, the cells are seeded in a medium in an appropriate environment in a small volume, followed by providing the desired stimulus, e.g., candidate compound, to provide the assay sample. As appropriate, for complex formation studies, a GPCR or protein of interest agonist is also added. The concentration of the agonist will be chosen to substantially optimize the assay.

The volume will generally not exceed about 250 μl, usually not more than about 200 μl, and generally be at least about 10 μl, more usually at least about 200 μl, where the volume of the candidate compound solution addition will generally dilute the cell medium less than about 1:1, usually not more than about 0.5:1. When the reagent is dry, there will be no dilution. After incubating the assay sample for sufficient time for the event of interest to occur, generally from about 0.1 h to about 0.5 day, enzyme substrate is added and the turnover of the substrate determined, usually by an optical method. Instead of having the substrate enter the cell, a reagent solution for lysis of the cells and containing a detectable β-galactosidase substrate may be added to the assay sample and one or more readings taken of the product from the substrate. The ratio of dilution will be not more than about 1:2, usually in the ratio of about 1:0.25 to 1:2, more usually 1:1 and as little at 1:0.25 or less. This dilution factor allows for reduced formation of complex during the reading period, while allowing for a robust signal, providing at least a five-fold, usually at least a 10-fold of ratio of signal to background during the period of the reading. One or more readings will be taken within 150 min, more usually within 120 min, preferably within about 60 min, and usually after about 10 min, more usually after about 15 min.

One interest is complexation of the arrestin to the GPCR at the membrane and, as appropriate, translocation of the GPCR from the membrane. There is substantially little, if any, formation of the active enzyme in the absence of stimulation of the GPCR and complexing with arrestin. Another interest as described above is complexation with a GPCR other than with the arrestin.

The transformed cells to be used in the assay will be treated conventionally, generally being grown in a complete medium, washed twice with PBS and then incubated in serum-free medium overnight. The media will be conventional for the particular cells used; F-12 for CHO cells, modified Eagle's media for U20S cells, standard DMEM for HEK cells, etc. The cells for use in the assay will be grown in accordance with the nature of the cells. For the most part, cells will be grown in wells in microtiter plates, the number of wells generally ranging from about 96 to 1536, generally being from 96 to 384 wells. The bottom will generally be clear, so that readings may be taken from the bottom of the wells. The number of cells plated in a well will generally range from about 10² to 10⁴ cells. The volume of the medium will usually be in the range of about 10 to 200 μl. The cells are then allowed to adhere overnight using conventional conditions of 37° C./5% CO₂.

After sufficient time for the stimulation of the cells to take effect from the candidate ligand and the arrestin to complex with the GPCR and the ED and EA to complex to form an active enzyme, one may add a substrate that is transported into the cell or a reagent solution is added for lysis of the cell, the reagent solution comprising a detectable substrate. With permeabilization or lysis, it is found that the formed enzyme complex is retained, the potential for new complex to form as a result of the permeabilizing of the cells is inhibited and the background from other than complex formed from the translocation is minimal. In this way a robust response to the activity of the stimulation is achieved. No further additions are required. A conventional commercially available optical plate reader can be used effectively.

The reagent solution provides for permeabilizing or lysis of the cells and release of any complex formed in the nucleus to the assay medium. Any conventional lysis buffer may be employed that does not interfere with the β-galactosidase reaction with its substrate. Various ionic buffers, such as CHAPS, may be employed at 1-5%, generally not more than 3%, in a convenient buffer, such as PBS and HEPES, where numerous other substitutes are known in the field.

Also present will be a β-galactosidase substrate, desirably a luminescent reagent and optionally a signal enhancer. The luminescent reagent will be in large excess in relation to the maximum amount of β-galactosidase that is likely to be formed. Conveniently, a luminescent substrate is used, available as Galacton Star from ABI in conjunction with the Emerald II enhancer. Any equivalent luminescent substrate composition may be employed. The substrate will be present in about 1 to 10 weight percent, while the enhancer will be present in about 10 to 30 weight percent of the reagent solution. These amounts will vary depending upon the particular substrate composition employed. The reagent solution may be prepared as a 5-20× concentrate or higher for sale or the solids may be provided as powders and dissolved in water at the appropriate proportions. Alternatively, a fluorescent substrate may be used and these have been extensively described in the patent, scientific and commercial literature.

Standards will usually be used, whereby the signal is related to the concentration of a known stimulator performed under the same conditions as the candidate compound. A graph can be prepared that shows the change in signal with the change in concentration of the standard compound. The assay is sensitive to EC₅₀ s of not greater than micromolar of candidate compound, generally sensitive to less than about 1 μM, in most cases sensitive to less than about 500 nM, frequently sensitive to less than 100 nM and can in many cases detect EC₅₀s of less than 50 nM. The S:B (signal/background) ratios are generally are at least about 2 fold, more usually at least about 3 fold, and can be greater than about 50 fold.

For convenience kits can be provided. In the subject assays, the EA fusion protein may be provided as a construct for expression of EA to be introduced into the cell or cells may be provided that are appropriately modified to provide EA in the cell. Generally, the kits would include an insert with instructions for performing the assay. The instructions may be printed or electronic, e.g., a CD or floppy disk. The kits find use in marketing the product and encouraging the use of the assay for research and commercial settings.

The following examples are offered by way of illustration and not by way of limitation.

### Experimental

The following exemplifies the work for determining the response to a ligand of a GPCR with or without the linker for phosphorylation.

The following are the sequences for the EDs PK1 and PK2, where the sequence for PK1 (SEQ ID NO: 11) is the first sequence listed below. Underlining and italicizing indicates the start and stop of the actual sequence of the PK1 and PK2. Additional amino acids indicate position of the sequence within an intact beta galactosidase protein.

H to R mutation shown between slashes

| PK (PK1) | DSLAVVLQRRDWENPGVTQLNRLAA/R/PPFASWRNSEEARTDRPSQQLR |
|---|---|
| | 10 20 30 40 50 |

No mutation

| | |
|---|---|
| PK2 | DSLAVVLQRRDWENPGVTQLNRLAAHPPFASWRNSEEARTDRPSQQLR |
| | 10 20 30 40 50 |

Preceding Sequence:
Flexible Linker:

GGGSGGGSLE (START PK sequence)

SEQ ID NOs: 11 and 12 illustrate PK1 and PK2, respectively; in this example, both sequences are preceded by the linker of SEQ ID NO:1. Thus, one protein described herein has the amino acid sequence of beta arrestin fused at its C terminus to an EA fragment having an amino acid sequence of beta galactosidase as given at world wide web address uniprot.org/uniprot/P00722, beginning with the non-underlined sequence above.

The ED used may be linked to the GPCR through an optional linker. The linker may be designed to include phosphorylation sites, such as serine or threonine phosphorylation sites. A number of proteins contain phosphorylation sequences, which may be, for example on the order of five to twenty amino acids long, and these may be engineered into he present linkers. This is done by known methods of altering the DNA encoding the construct by restriction enzyme digestion and ligation of the desired DNA sequence encoding the amino acid sequence desired

The genes for the GPCRs may be obtained from any convenient source: commercial supplier; RT_PCR from mRNA isolated in accordance with conventional procedures using known sequences as probes; PCR from genomic DNA using primers from known sequences. The genes are PCR amplified to remove the stop codon at the 3′ end. The genes are then digested with restriction enzymes where the restriction site is included within the primer sequences. These products are then purified in conventional ways and then ligated into a commercial vector into which the ED or EA has been inserted in reading frame with the ED or EA. Separating the ED and the EA from the gene is a gly-ser linker that provides flexibility to the fusion proteins to enhance complementation. This linker is not required for activity. When the additional linker having a phosphorylation sequence is included, this sequence will be fused to the gene at one end and the indicated gly-ser linker at the other end to provide for a fusion protein having in the N-C direction, the gene, optionally the phosphorylation sequence, the linker and the ED. The transcriptional regulatory region is generally present in commercial vectors, such as the 5′ LTR of the virus used for the vector. Alternatively, the CMV promoter may be used. The resulting vector is then introduced into the host cell by liposome mediated transfection or retroviral infection with Moloney murine leukemia virus vector and packaging cell lines. The resulting virus is then used for viral infection. The vectors also include selection genes, such as hygromycin resistance and cells into which the construct is integrated are selected in a conventional selection medium. The surviving cells are then screened in an agonist dose response assay using adherent cells and the Path-Hunter® Detection Kit reagents in white-walled microplates.

To perform the screening assay, 20 μl of cells in complete media or serum-free media (OPTI-MEM® Invitrogen Cat. #31985-070) were plated at 5, 10 and 15×10³ cells per well in a white 384 well microplate and incubated at 37° C. overnight. Cells are allowed to adhere and grown overnight. Cells may be screened for their response in serum free or complete medium. Serial dilutions are then performed in serum-free medium or buffer for a volume of 25 μl. Depending on the candidate compound, the dilution may occur in the presence of 0.1% BSA or ≦1% final concentration of an organic solvent, e.g., DMSO and methanol. 5 μl of each candidate compound is added per well. The assay mixture is then incubated for 90 min at 37° C. 12.5 μl of the detection reagent is then added. The detection reagent is prepared by combining 1 part Galacton Star Substrate® with 5 parts Emerald II® Solution and 19 parts Path-Hunter Cell Assay Buffer (lysis buffer). The assay mixture is then incubated for 1 h at room temperature. Chemiluminescence is then read at 1.0 sec/well on PMT based instrument or 5-20 sec on an imager.

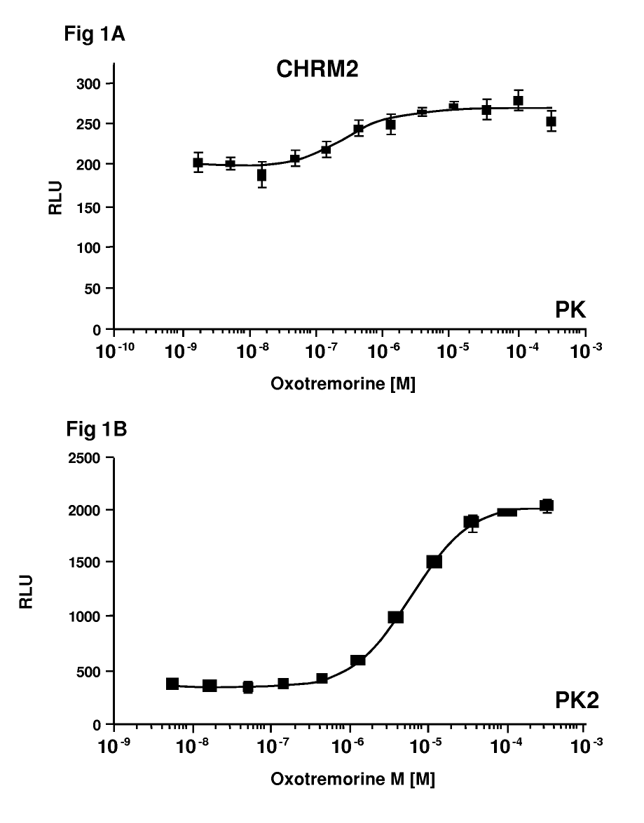

In association with FIG. 1, the following table indicates the signal to background ratios (S:B) with a number of different GPCRs labeled with PK1 (SEQ ID NO:11) or PK2 (SEQ ID NO:12). Receptors are identified by their gene symbols. CCCR4 is chemokine receptor 4; CHRM2 is cholinergic receptor, muscarinic 2; CRHR2 is corticotropin releasing hormone receptor 2; CRTH2 is G Protein-coupled receptor 44; MC3R is melanocortin 3 receptor; and OPRM1 is opiod receptor mu-1. SSTR1 is somatostatin receptor 1; SSTR4 is somatostatin receptor 4; HRH3 is histamine receptor H3.

| RECEPTOR | PK1 S:B | PK2 S:B |
|---|---|---|
| CCR4 | 19 | 3 |
| CHRM2 | 1.6 | 6.1 |
| CRHR2 | 13 | 17 |
| CRTH2 | 1 | 2.6 |
| MC3R | 1 | 3.2 |
| OPRM1 | 26 | 8 |

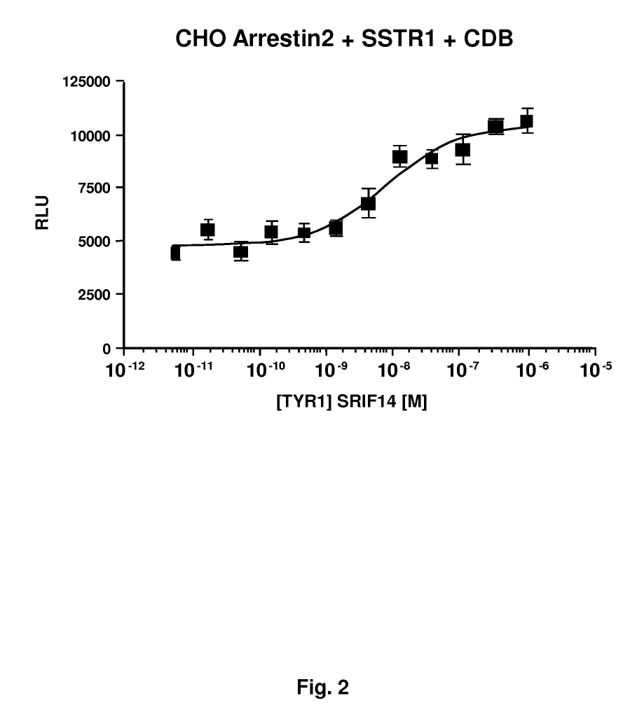

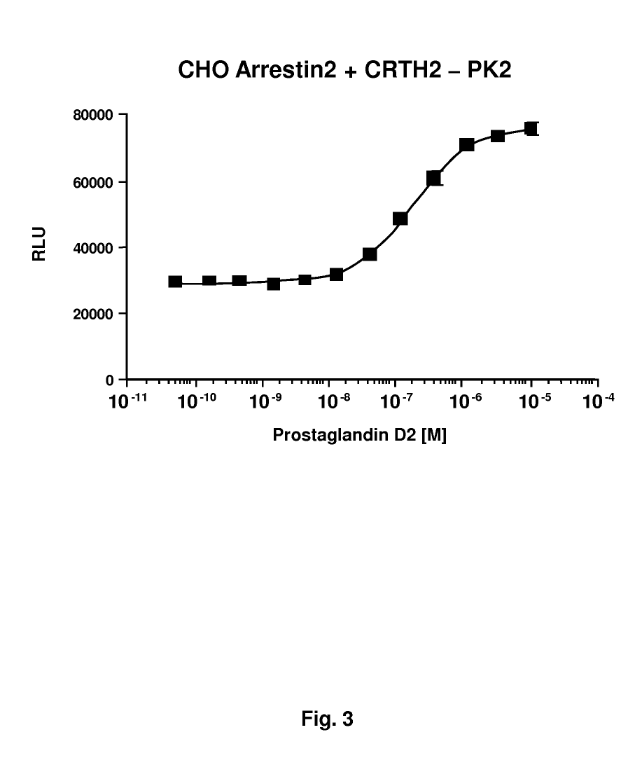

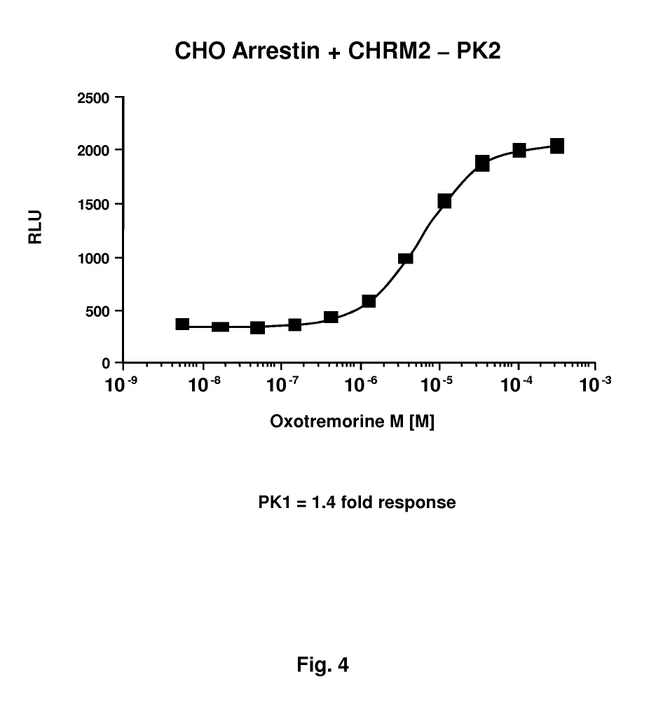

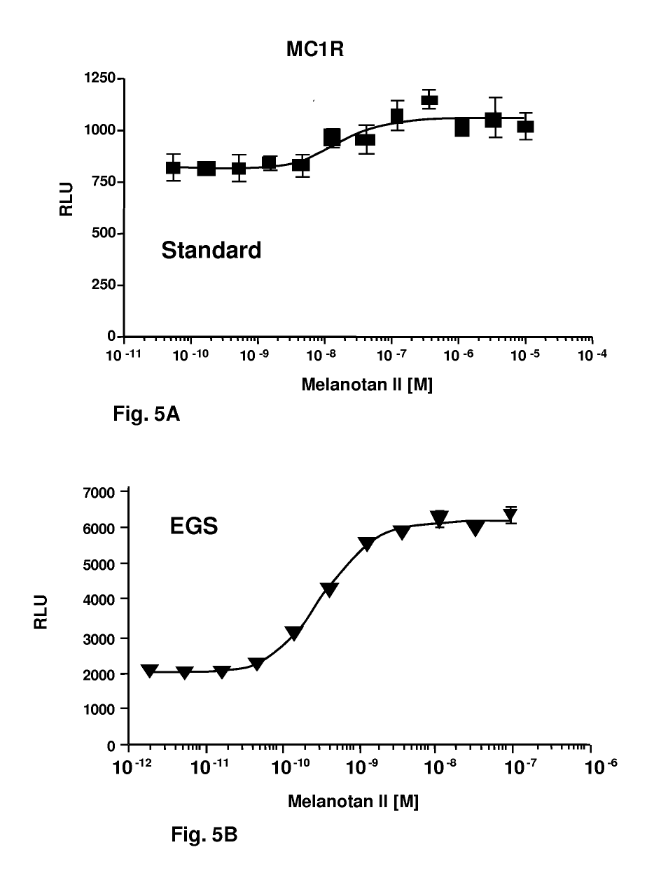

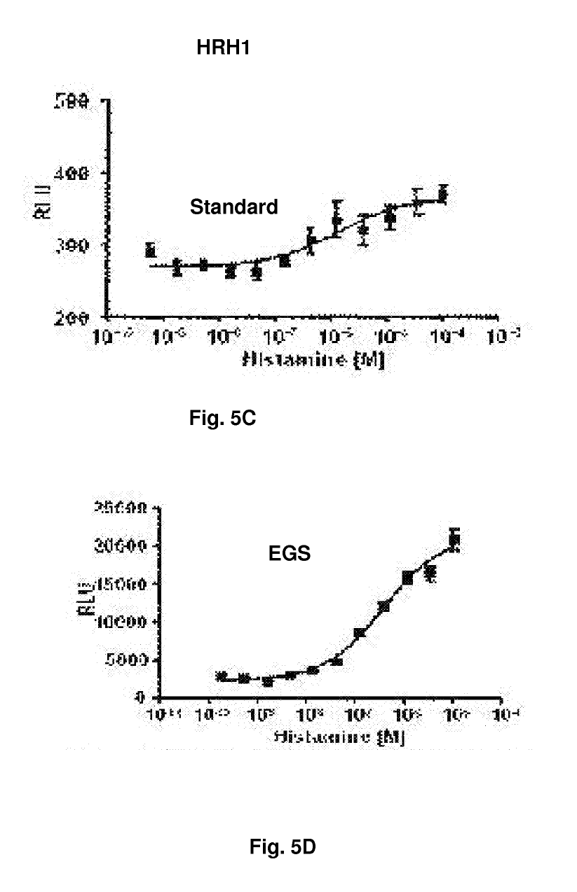

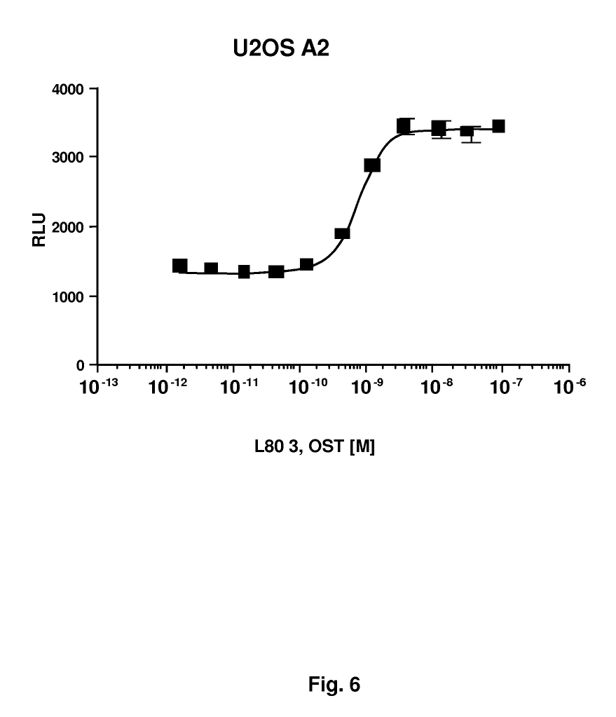

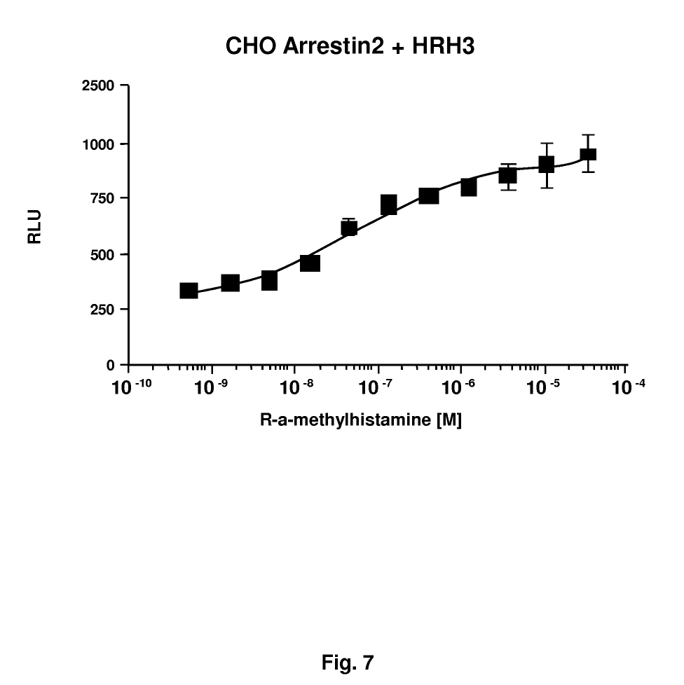

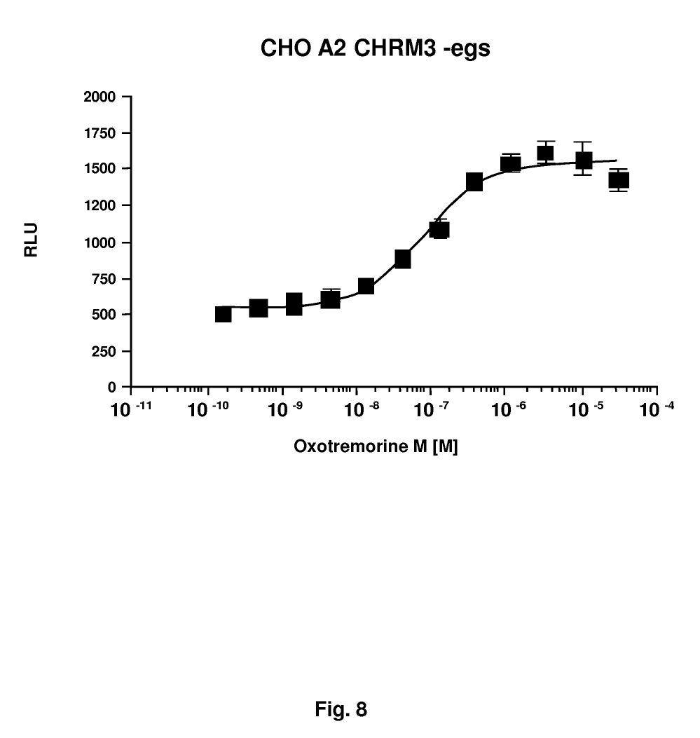

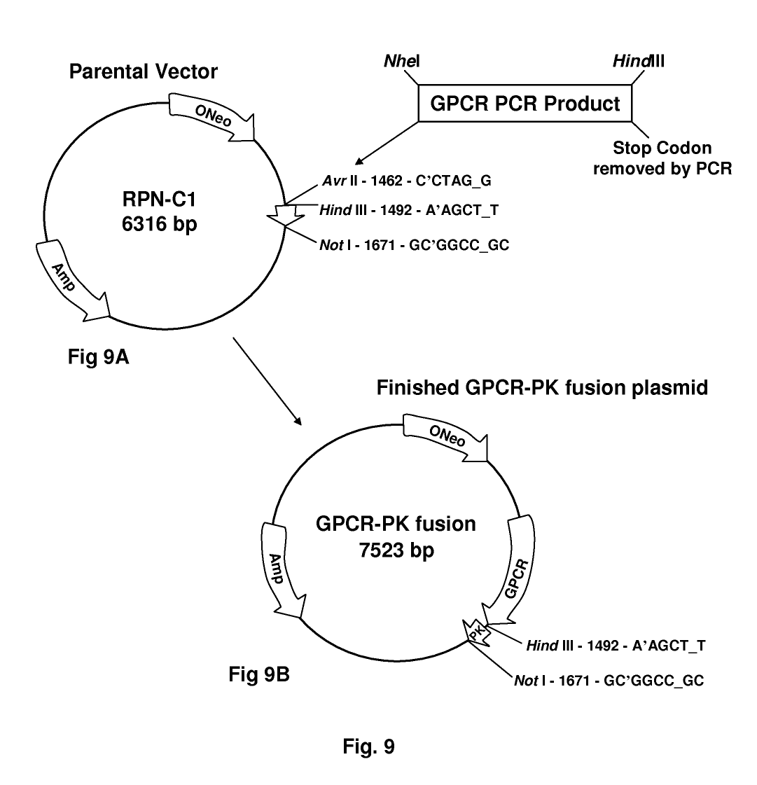

For FIGS. 2-4 and 5-7 the results tabulated for the graph are set forth in the following table:

| | SSTR1 | CRTH2 | CHRM2 | SSTR4 | HRH3 | CHRM3 |
|---|---|---|---|---|---|---|
| Bottom | 4794 | 29223 | 335.9 | 1380 | 273.1 | |
| Top | 10484 | 75978 | 2037 | 3418 | 934.9 | |
| Log EC₅₀ | −8.069 | −6.760 | −5.201 | −9.136 | −7.258 | |
| Hill Slope | 0.7893 | 1.018 | 1.183 | 2.138 | 0.5419 | |
| EC₅₀ | 8.1506e⁻⁰⁰⁹ | 1.7376e⁻⁰⁰⁷ | 6.2881e⁻⁰⁰⁶ | 7.3187e⁻⁰¹⁰ | 5.5194e⁻⁰⁰⁸ | |
| PK1/PK2 | 2 | 2 | 2 | ? | ? | |

The following exemplifies the work demonstrating the determination of transactivation of a heterooligomer, usually dimer, comprising a GPCR non-covalently bound to a protein of interest, which may be a receptor, such as a different GPCR.

Exemplary nucleic Acid sequence of the GPCR (OPRD1)-PK Fusion protein:

(SEQ ID NO: 13)

ATGGAACCGGCCCCCTCCGCCGGCGCCGAGCTGCAGCCCCCGCTCTTCGC
CAACGCCTCGGACGCCTACCCTAGCGCCTGCCCCAGCGCTGGCGCCAATG
CGTCGGGGCCGCCAGGCGCGCGGAGCGCCTCGTCCCTCGCCCTGGCAATC
GCCATCACCGCGCTCTACTCGGCCGTGTGCGCCGTGGGGCTGCTGGGCAA
CGTGCTTGTCATGTTCGGCATCGTCCGGTACACTAAGATGAAGACGGCCA
CCAACATCTACATCTTCAACCTGGCCTTAGCCGATGCGCTGGCCACCAGC
ACGCTGCCTTTCCAGAGTGCCAAGTACCTGATGGAGACGTGGCCCTTCGG
CGAGCTGCTCTGCAAGGCTGTGCTCTCCATCGACTACTACAATATGTTCA
CCAGCATCTTCACGCTCACCATGATGAGTGTTGACCGCTACATCGCTGTC
TGCCACCCTGTCAAGGCCCTGGACTTCCGCACGCCTGCCAAGGCCAAGCT
GATCAACATCTGTATCTGGGTCCTGGCCTCAGGCGTTGGCGTGCCCATCA
TGGTCATGGCTGTGACCCGTCCCCGGGACGGGGCAGTGGTGTGCATGCTC
CAGTTCCCCAGCCCCAGCTGGTACTGGGACACGGTGACCAAGATCTGCGT
GTTCCTCTTCGCCTTCGTGGTGCCCATCCTCATCATCACCGTGTGCTATG
GCCTCATGCTGCTGCGCCTGCGCAGTGTGCGCCTGCTGTCGGGCTCCAAG
GAGAAGGACCGCAGCCTGCGGCGCATCACGCGCATGGTGCTGGTGGTTGT
GGGCGCCTTCGTGGTGTGTTGGGCGCCCATCCACATCTTCGTCATCGTCT
GGACGCTGGTGGACATCGACCGGCGCGACCCGCTGGTGGTGGCTGCGCTG
CACCTGTGCATCGCGCTGGGTTACGCCAATAGCAGCCTCAACCCCGTGCT
CTACGCTTTCCTCGACGAGAACTTCAAGCGCTGCTTCCGCCAGCTCTGCC
GCAAGCCCTGCGGCCGCCCAGACCCCAGCAGCTTCAGCCGCGCCCGCGAA
GCCACGGCCCGCGAGCGTGTCACCGCCTGCACCCCGTCCGATGGTCCCGG
CGGTGGCGCTGCCGCCATAAGCTTCGAATTGGGAGGTGGCGGTAGCGGAG
GTGGCGGTAGCCTCGAGGATTCACTGGCCGTCGTTTTACAACGTCGTGAC
TGGGAAAACCCTGGCGTTACCCAACTTAATCGCCTTGCAGCACGTCCCCC
TTTCGCCAGCTGGCGTAATAGCGAAGAGGCCCGCACCGATCGCTGA

Exemplary Amino Acid sequence of the OPRD1-PK Fusion

MEPAPSAGAELQPPLFANASDAYPSACPSAGANASGPPGARSASSLALAI
AITALYSAVCAVGLLGNVLVMFGIVRYTKMKTATNIYIFNLALADALATS
TLPFQSAKYLMETWPFGELLCKAVLSIDYYNMFTSIFTLTMMSVDRYIAV
CHPVKALDFRTPAKAKLINICIWVLASGVGVPIMVMAVTRPRDGAVVCML
QFPSPSWYWDTVTKICVFLFAFVVPILIITVCYGLMLLRLRSVRLLSGSK
EKDRSLRRITRMVLVVVGAFVVCWAPIHIFVIVWTLVDIDRRDPLVVAAL
HLCIALGYANSSLNPVLYAFLDENFKRCFRQLCRKPCGRPDPSSFSRARE
ATARERVTACTPSDGPGGGAAAISFELGGGGSGGGGSLEDSLAVVLQRRD
WENPGVTQLNRLAARPPFASWRNSEEARTDR.

### (SEQ ID NO: 14)

In the above sequence, the linker is italicized, and PK is in Bold and underlined.

### Assay Protocols

For all assays, 10,000 cells per well were seeded in 20 μL media and incubated overnight in 1% Fetal Bovine Serum and appropriate basal media (F-12 or DMEM). For agonist assays, 5 μL compound was added to cells and incubated at Room Temp. For antagonist assays, 5 μL 5× compound was added to cells and incubated at 37° C./5% CO₂ for 10 minutes, after which 5 μL 6× agonist was added and incubated for 60 minutes at 37° C. Arrestin-EA complex formation with the GPCR was detected with 50% (v/v) of PathHunter Detection Reagent (Dx 93-0001, PathHunter reagents are available from DiscoveRx, Corp., Fremont, Calif.) (Lysis buffer active ingredient 2% CHAPS, Emerald II and Galacton star are from Applied Biosystems.) Data was read on Packard Victor 2 or PerkinElmer ViewLux readers and analyzed using GraphPad Prism 4.

In all of the cell lines tested, arrestin-EA is being expressed along with the other proteins that are being studied.

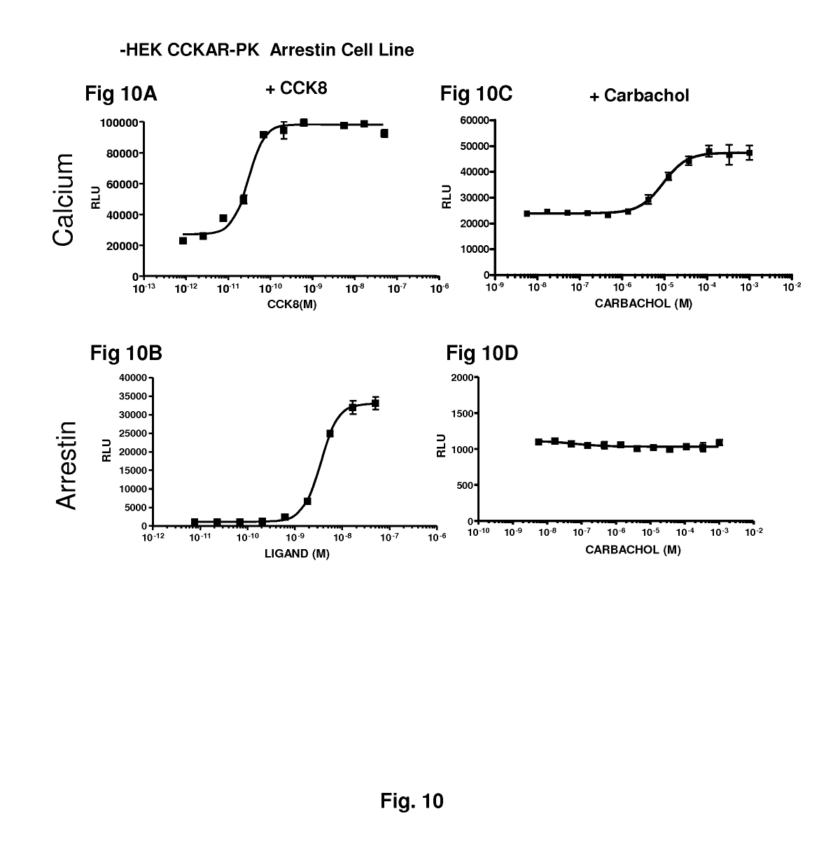

Turning to the Figures, FIG. 10 shows the specificity of the subject assay for the CCD8 ligand binding to the fusion protein CCKAR-PK. In the upper and lower graphs (A and B), CCK8 is added to a cell line expressing CCKAR-PK and arrestin-EA. It is noted that the calcium increase resulting from the binding of CCK8 to CCKAR parallels the result observed for the arrestin-EA binding to the CCKAR-PK. However, for the non-ligand carbachol, while there is some effect on calcium increase, there is substantially no effect on the binding of arrestin-EA to CCKAR-PK.

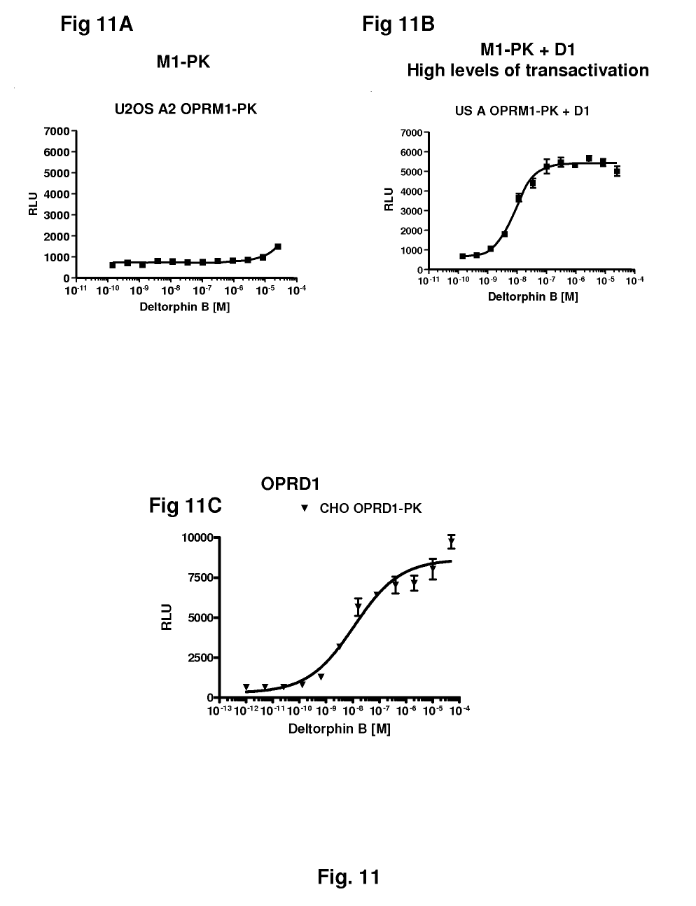

FIG. 11 has graphs demonstrating transactivation using cells having the GPCR OPRD1 (opiod receptor delta 1) and the fusion protein of the GPCR OPRM1 (opiod receptor mu-1) fused to PK. In A), adding the D1 ligand, deltorphin (a known delta opiod receptor agonist) to a cell expressing only M1-PK has substantially no effect on arresting-EA binding. In B), a cell co-expressing OPRM1-PK and OPRD1, there is a substantial response when dextorphin binds to the OPRD1, demonstrating that in the complex of OPRM1 and OPRD1 in the membrane, when OPRD1 is activated by deltorphin, arrestin-EA binds to the OPRM1-PK resulting in formation of a functional β-galactosidase, which can be detected with an appropriate substrate.

Example illustrates binding of a GPCR of one type to t protein that is another type of GPCR.

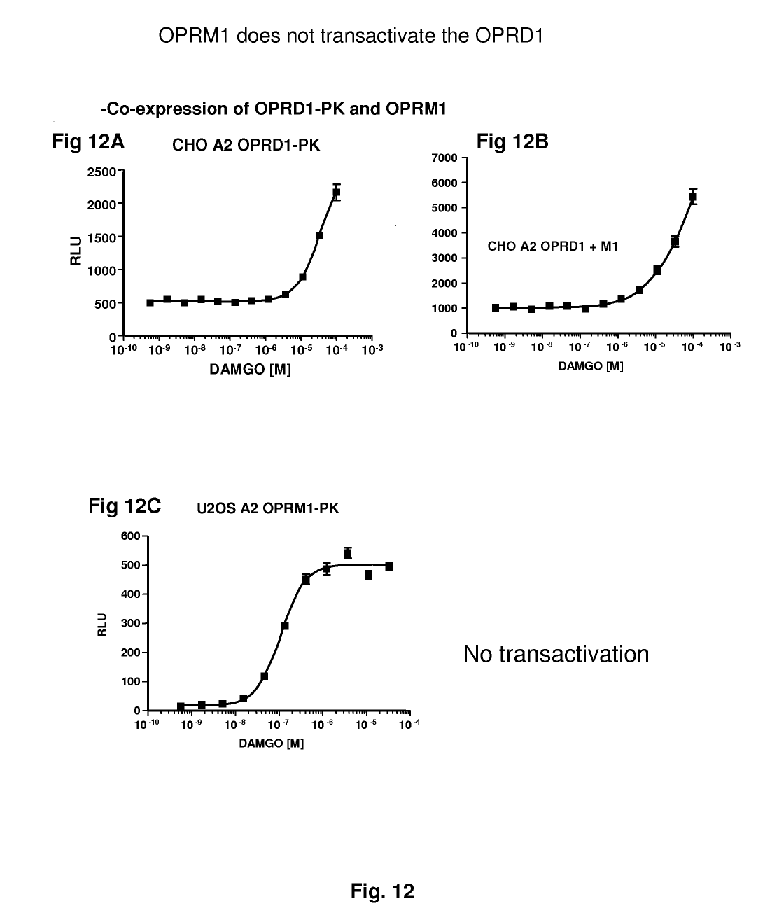

FIG. 12 shows that transactivation is not universal and not all GPCRs that form complex pairs provide for transactivation. Using the cell line CHO A2 or U20S as the cellular hosts, cells expressing only OPRD1-PK, OPRD1-PK and OPRM1 and only OPRM1-PK are treated with DAMAGO, a ligand for OPRM1. DAMAGO is a known specific μ opioid receptor agonist, [D-Ala2,N-Me-Phe4,Gly5-ol]-enkephalin. The upper and lower left hand graphs compare the absence of response of OPRD1-PK and the presence of response of OPRM1-PK, respectively. The right hand graph tracks with the lack of response of OPRD1-PK.

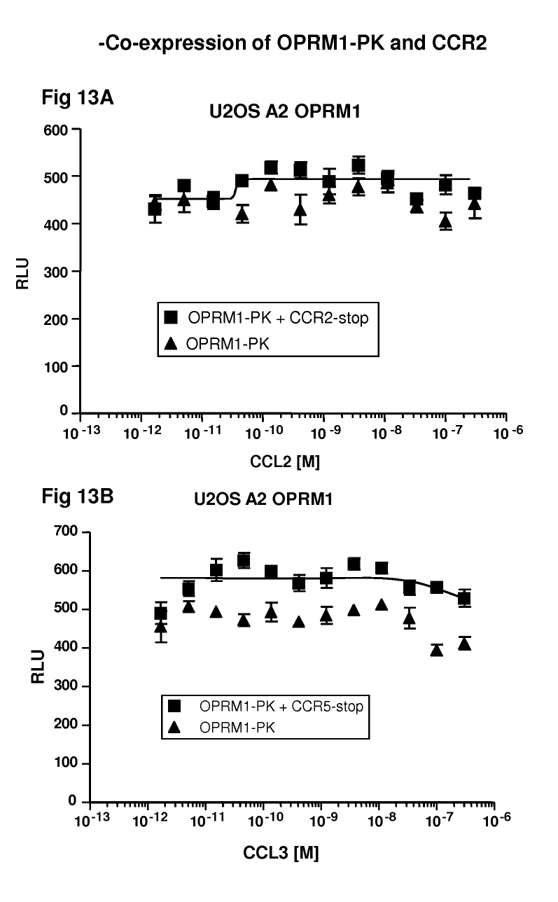

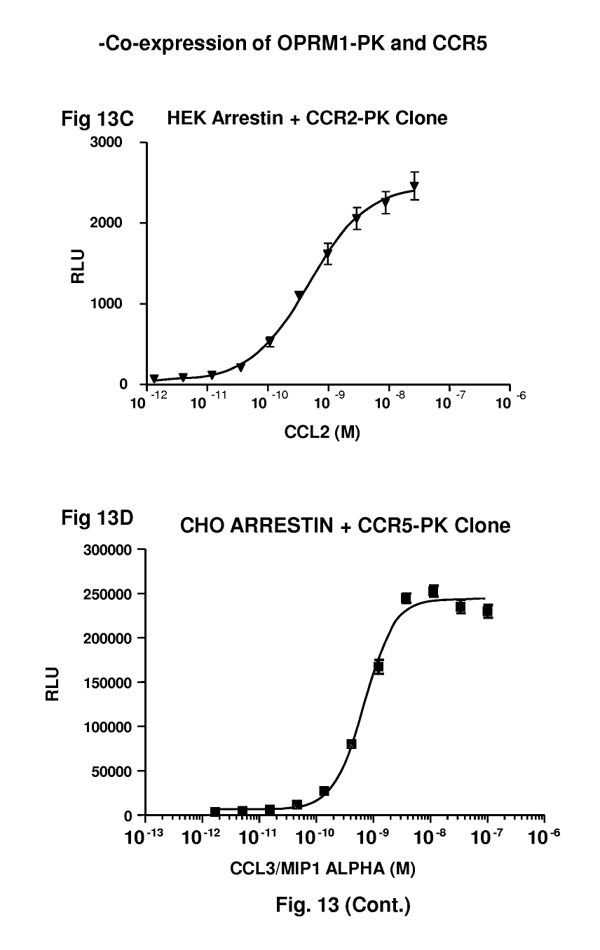

FIG. 13 also shows the lack of transactivation between receptors CCR2 or CCR5 coexpressed with ORPM1-PK. As graphs A and B show there is no response when the ligand CCL3 is added to the cells. However, when CCLs is added to CCR2-PK or CCR5-PK expressing cells there is a substantial response with variation in concentration.

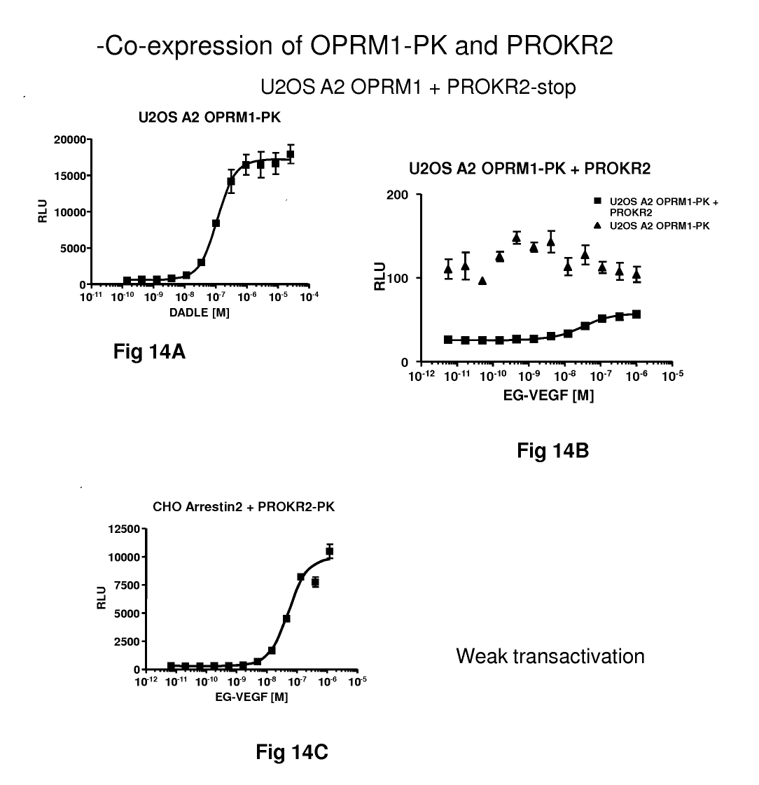

FIG. 14 demonstrates that by contrast with the robust response observed with OPRD1 and OPRM1-PK, with other protein receptors a weak but observable response is obtained. When OPRM1-PK and PROKR2 are coexpressed, a weak response is observed upon addition of the PROKR2 ligand EG-VEGF. The upper and lower left hand graphs show the responses of OPRM1-PK and PROKR2-PK to their respective ligands, while the right hand graph shows the response of cells coexpressing OPRM1-PK and PROKR2. See Masuda Y, Takatsu Y, Terao Y, Kumano S, Ishibashi Y, et al. (2002) Isolation and identification of EG-VEGF/prokineticins as cognate ligands for two orphan G-protein-coupled receptors. Biochem Biophys Res Commun 293: 396-402.

It is evident from the above results that by providing for an additional phosphorylation site on the GPCR, a substantially improved assay for candidate compounds binding to the GPCR is achieved. Improved signal to noise ratios are obtained, so as to provide for higher sensitivity for detection of active compounds. By virtue of the higher sensitivity, one obtains a broader range of activities, so as to be able to establish lower active concentrations for candidate compounds.

Although the invention has been described with reference to the above examples, it will be understood that modifications and variations are encompassed within the spirit and scope of the invention. Accordingly, the invention is limited only by the following claims. All references referred to in the specification are incorporated by reference as if fully set forth therein.

What is claimed is:

1. A method for determining G-coupled protein cellular receptor (“GPCR”) activation, employing β-galactosidase enzyme fragment complementation using an enzyme acceptor fragment (“EA”) and an enzyme donor fragment (“ED”), comprising the steps of:
(a) providing a first fusion protein comprising (a) a GPCR or a GPCR binding protein linked to said ED;
(b) providing a second fusion protein comprising arrestin linked to said EA, where, when said arrestin is bound to said GPCR or a GPCR binding protein, a functional β-galactosidase is formed; and
(c) providing cells transformed with genetic constructs expressing said first and second fusion proteins, said method further comprising the steps of:
i. incubating said cells in an assay medium in a selected environment for sufficient time for any binding to occur, said environment comprising one or both of said GPCR ligand and GPCR binding protein;
ii. adding a β-galactosidase substrate, which substrate results in a detectable signal; and
iii. determining said signal as a measure of said binding.

2. A method according to claim 1, wherein said signal is a chemiluminescent signal.

3. A method according to claim 1, wherein said ED is a low affinity small fragment mutated from the natural β-galactosidase sequence.

4. A method according to claim 1 wherein said method comprises (a).

5. A method according to claim 4, wherein said ED has SEQ ID NO: 10 (PK1).

6. A method according to claim 1, wherein said method comprises (b).

7. A method according to claim 6, wherein said selected environment comprises an agonist for said GPCR and a candidate compound for modulating said protein of interest binding to said GPCR.

8. A method for screening binding of a GPCR to a candidate GPCR ligand employing β-galactosidase enzyme fragment complementation assay, using an enzyme donor fragment (“ED”) and an enzyme acceptor fragment (“EA”), a first fusion protein comprising a GPCR linked to a fragment of β-galactosidase (“ED”) joined to a sequence comprising a naturally occurring GPCR phosphorylation site or a consensus sequence of naturally occurring GPCR phosphorylation sites, which links to an enzyme donor fragment (“ED”) and a second fusion protein comprising arrestin linked to the complementary fragment of β-galactosidase (“EA”), where when said arrestin is bound to said GPCR a functional β-galactosidase is formed, and the ED, EA and said linker are selected to provide binding of said GPCR and arrestin to provide a substantially optimized signal, employing cells transformed with genetic constructs expressing said first and second fusion proteins, said method comprising:
a. incubating said cells in an assay medium in a selected environment for sufficient time for any binding to occur;
b. adding a β-galactosidase substrate, which substrate results in a detectable signal; and
c. determining said signal as a measure of said binding.

9. A method according to claim 8, wherein said ED is a low affinity small fragment mutated from the natural β-galactosidase sequence.

10. A method according to claim 8, wherein said ED is a high affinity small fragment.

11. A method for screening binding of a GPCR to a protein of interest employing β-galactosidase enzyme fragment complementation assay, using an enzyme donor fragment (“ED”) and an enzyme acceptor fragment (“EA”), a first fusion protein with ED fused to said protein of interest or to said GPCR, and a second fusion protein comprising arrestin linked to the complementary fragment of β-galactosidase (“EA”), where when said arrestin is bound to said GPCR a functional β-galactosidase is formed, employing cells comprising said GPCR and transformed with genetic constructs expressing said first and second fusion proteins, with the proviso that when said ED is fused to said GPCR agonist added binds to said protein of interest, said method comprising:
a. incubating said cells in an assay medium comprising an agonist and a candidate compound for modulating said binding for sufficient time for any binding to occur;
b. adding a β-galactosidase substrate, which substrate results in a detectable signal; and
c. determining said signal as a measure of said binding.

12. A method according to claim 11, wherein said agonist binds to said protein of interest.

13. A method according to claim 11, wherein said agonist binds to said GPCR.

14. A method according to claim 11, wherein said ED is fused to said GPCR.

15. A method according to claim 11, wherein said ED is fused to said protein of interest and said agonist binds to said GPCR.


SEQUENCE LISTING (14 sequences)

SEQ ID NO: 1 - protein, 10 aa, artificial
  GPCR phosphylation recognition site
GGGSGGGSLE

SEQ ID NO: 2 - protein, 16 aa, artificial
  GPCR phosphorylation site
SYNGSKXSPASLSRFS

SEQ ID NO: 3 - protein, 9 aa, artificial
  GPCR phosphorylation site
SASYXSGHS

SEQ ID NO: 4 - protein, 14 aa, artificial
  GPCR recogniztion site
CASLSRFSYSHYMS

SEQ ID NO: 5 - protein, 13 aa, artificial
  GPCR phosphorylation site
IASLSRLSYTTIS

SEQ ID NO: 6 - protein, 9 aa, artificial
  GPCR phosphorylation site
SQRSCSQPS

SEQ ID NO: 7 - protein, 7 aa, artificial
  GPCR phosphorylation site
RSLXSCS

SEQ ID NO: 8 - protein, 8 aa, artificial
  GPCR phosphorylation site
DDSGSCLS

SEQ ID NO: 9 - protein, 8 aa, artificial sequence
  GPCR phosphorylation site
SYSHMSAS

SEQ ID NO: 10 - protein, 8 aa, artificial sequence
  GPCR phosphorylation site
SYTTISTL

SEQ ID NO: 11 - protein, 48 aa, artificial
  beta galactosidase ED fragment
DSLAVVLQRRDWENPGVTQLNRLAARPPFASWRNSEEARTDRPSQQLR

SEQ ID NO: 12 - protein, 48 aa, artificial sequence
  beta galactosidase ED fragment
DSLAVVLQRRDWENPGVTQLNRLAAHPPFASWRNSEEARTDRPSQQLR

SEQ ID NO: 13 - DNA, 1296 bp, artificial
  GPCR - beta lactamase ED fragment fusion protein
atggaaccggccccctccgccggcgccgagctgcagcccccgctcttcgccaacgcctcggacgcctaccctagcgcctgccccagcgctggcgccaatgcgtcggggccgccaggcgcgcggagcgcctcgtccctcgccctggcaatcgccatcaccgcgctctactcggccgtgtgcgccgtggggctgctgggcaacgtgcttgtcatgttcggcatcgtccggtacactaagatgaagacggccaccaacatctacatcttcaacctggccttagccgatgcgctggccaccagcacgctgcctttccagagtgccaagtacctgatggagacgtggcccttcggcgagctgctctgcaaggctgtgctctccatcgactactacaatatgttcaccagcatcttcacgctcaccatgatgagtgttgaccgctacatcgctgtctgccaccctgtcaaggccctggacttccgcacgcctgccaaggccaagctgatcaacatctgtatctgggtcctggcctcaggcgttggcgtgcccatcatggtcatggctgtgacccgtccccgggacggggcagtggtgtgcatgctccagttccccagccccagctggtactgggacacggtgaccaagatctgcgtgttcctcttcgccttcgtggtgcccatcctcatcatcaccgtgtgctatggcctcatgctgctgcgcctgcgcagtgtgcgcctgctgtcgggctccaaggagaaggaccgcagcctgcggcgcatcacgcgcatggtgctggtggttgtgggcgccttcgtggtgtgttgggcgcccatccacatcttcgtcatcgtctggacgctggtggacatcgaccggcgcgacccgctggtggtggctgcgctgcacctgtgcatcgcgctgggttacgccaatagcagcctcaaccccgtgctctacgctttcctcgacgagaacttcaagcgctgcttccgccagctctgccgcaagccctgcggccgcccagaccccagcagcttcagccgcgcccgcgaagccacggcccgcgagcgtgtcaccgcctgcaccccgtccgatggtcccggcggtggcgctgccgccataagcttcgaattgggaggtggcggtagcggaggtggcggtagcctcgaggattcactggccgtcgttttacaacgtcgtgactgggaaaaccctggcgttacccaacttaatcgccttgcagcacgtccccctttcgccagctggcgtaatagcgaagaggcccgcaccgatcgctga

SEQ ID NO: 14 - protein, 431 aa, artificial
  GPCR - beta lactamase ED fragment fusion protein
MEPAPSAGAELQPPLFANASDAYPSACPSAGANASGPPGARSASSLALAIAITALYSAVCAVGLLGNVLVMFGIVRYTKMKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTRPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDIDRRDPLVVAALHLCIALGYANSSLNPVLYAFLDENFKRCFRQLCRKPCGRPDPSSFSRAREATARERVTACTPSDGPGGGAAAISFELGGGGSGGGGSLEDSLAVVLQRRDWENPGVTQLNRLAARPPFASWRNSEEARTDR